(19) United States
(12) Patent Application Publication
(10) Pub. No.: US 2012/0225028 A1
(43) Pub. Date: Sep. 6, 2012

(54) COMPOSITIONS AND METHODS FOR MOBILIZATION OF STEM CELLS

(76) Inventors: Moshe Cohen, West Orange, NJ (US); Ian Keith McNiece, Coral Gables, FL (US)

(21) Appl. No.: 13/411,265

(22) Filed: Mar. 2, 2012

Related U.S. Application Data
(60) Provisional application No. 61/448,371, filed on Mar. 2, 2011.

(51) Int. Cl. A61K 38/19; A61P 9/00; C12N 5/071; A61P 13/12; A61P 11/00; A61P 29/00; A61P 25/28; A61P 1/16

(52) U.S. Cl. 424/85.1; 435/325

(57) ABSTRACT

The present invention relates to mobilization of stem cells. In particular, the present invention relates to growth factor-induced mobilization of stem cells in vivo for collection or tissue repair.

The present application claims priority to U.S. Provisional Patent Application Ser. No. 61/448,371, filed Mar. 2, 2011, the disclosure of which is herein incorporated by reference in its entirety.

### FIELD OF THE INVENTION

The present invention relates to mobilization of stem cells. In particular, the present invention relates to growth factor-induced mobilization of stem cells in vivo for collection or tissue repair.

### BACKGROUND OF THE INVENTION

Cellular therapy offers the potential of tissue repair for a number of diseases. However, there are many outstanding issues. These include the source of cells for therapeutic use, the mode of delivery, integration of the cells into the damaged tissue and the potential of the cellular product to regenerate functional tissue. The ideal cellular product would be an autologous product, eliminating graft versus host or rejection issues that could be delivered in the peripheral circulation. Cytokine-induced cell mobilization offers a readily available autologous cell source which can be delivered in a non-invasive manner. However, despite exciting preclinical data in animal models, the clinical studies to date have shown minimal clinical benefit. These studies have utilized granulocyte colony stimulating factor (G-CSF), which is routinely used clinically for mobilization of hematopoietic stem (HSC) and progenitor (HPC) cells. Many reports have shown that treatment with G-CSF leads to egress of HSC and HPCs from the bone marrow (BM) to the peripheral circulation. An explanation for the lack of efficacy in the clinical studies is that G-CSF mobilization mobilizes HSCs and HPCs which do not have the capacity to differentiate into other tissue specific lineages.

The art is in need of improved methods for mobilization of stem cells that are able to differentiate into specific tissues.

### SUMMARY OF THE INVENTION

The present invention relates to mobilization of stem cells. In particular, the present invention relates to growth factor-induced mobilization of stem cells in vivo for collection or tissue repair.

Embodiments of the present invention provide improved methods for mobilizing stem cells (e.g., from the bone marrow) into circulation. Improved mobilization of stem cells finds use in a variety of research, clinical and pharmaceutical applications (e.g., harvesting of stem cells or treatment of diseases of degeneration or inflammation) that are important for human or animal health, as well as diagnostics, drug discovery, and research applications.

For example, in some embodiments, the present invention provides a stem cell mobilization composition, comprising: a) at least one cytokine; and b) an immunostimulant (e.g., Plerixafor). In some embodiments, the cytokine is granulocyte colony-stimulating factor (G-CSF) (e.g., rhG-CSF). In some embodiments, the composition comprises or further comprises at least one additional stem cell mobilization reagent such as a neuropeptide (e.g., substance P such as that described by SEQ ID NO:1). In some embodiments, the composition comprises a) rhG-CSF and b) Plerixafor and c) substance P. In some embodiments, the composition is a pharmaceutical composition comprising a pharmaceutically acceptable carrier.

Further embodiments provide a method of mobilizing stem cells (e.g., mesenchymal stem cells), comprising: administering a stem cell mobilization composition, comprising: a) at least one cytokine; and b) at least one additional stem cell mobilization reagent to a subject under conditions such that the subject mobilizes circulating stem cells. In some embodiments, the method further comprises the step of isolating the stem cells from the subject. In some embodiments, the subject exhibits symptoms of a neurodegenerative, cardiac or inflammatory disease and mobilization of stem cells reduces the symptoms. In some embodiments, the stem cells are not hematopoietic stem cells. In some embodiments, the subject has a liver, kidney, neural, pulmonary, skin or blood diseases (e.g., due to tissue damage). In some embodiments, the subject has tissue damage due to an inflammatory response (e.g., in myocardial infarction, graft versus host disease and stroke). In some embodiments, the subject has undergone an organ transplant (e.g., liver, kidney, lung, heart, pancreas, etc.).

Additional embodiments provide the use of a composition, comprising: a) at least one cytokine; and b) an immunostimulant (e.g., Plerixafor) in a medicament (e.g., for treatment of a neurodegenerative, cardiac or inflammatory disease).

In some embodiments, the methods and pharmaceutically acceptable compositions described herein are used to treat a subject having a condition associated with one or more of the diseases or conditions described herein but are not used to treat one or more of the following diseases or conditions or are lacking at least one (or multiple or all) of the following diseases or conditions or are not in need of treatment of at least one (or multiple or all) of the following diseases or conditions: mobilization of hematopoietic stem cells, for example, in patients with multiple myeloma, non-Hodgkin's lymphoma, and Hodgkin's disease.

In some embodiments, the methods and compositions are used to treat subjects not in need of mobilization of hematopoietic stem cells, in particular patients with lymphoma (e.g., Hodgkin's or non-Hodgkin's lymphoma) or leukemia (e.g., multiple myeloma) not in need of mobilization of hematopoietic stem cells. In some embodiments, the methods and compositions are used to treat subjects lacking lymphoma (e.g., Hodgkin's or non-Hodgkin's lymphoma) or leukemia (e.g., multiple myeloma).

In some embodiments, the methods and pharmaceutically acceptable compositions provided herein are not used to treat one or more of the following cancers or are used to treat subjects who do not have at least one of the following cancers: lymphoma (e.g., Hodgkin's or non-Hodgkin's lymphoma) or leukemia (e.g., multiple myeloma).

Additional embodiments are described herein.

### DESCRIPTION OF THE FIGURES

FIG. 1 shows culture of normal (left panel) or mobilized peripheral blood (right panel) WBCs under MSC conditions.

FIG. 2 shows culture of peripheral blood WBCs from mice injected with saline (left panel) or rhG-CSF (right panel).

FIG. 3 shows stromal cell formation from WBC from mice treated with SP (left panel). The WBC from SP treated animals contained increased numbers of blast cells (right panel).

FIG. 4 shows MSC formation from peripheral blood WBC from mice treated with SP (left panel), G-CSF (middle panel) or the combination of SP+rhG-CSF (right panel).

FIG. 5 shows MSC formation at 3 weeks of culture from WBC from mice treated with G-CSF (left panel) or SP+G-CSF (right panel).

FIG. 6 shows a typical hematopoietic colony (left panel) and mixed hematopoietic cell and MSC colony from SP or rhG-CSF+SP treated mice.

FIG. 7 shows peripheral blood WBC counts for mice treated with G-CSF, SP or SP+G-CSF for 7 days.

FIG. 8 shows peripheral blood WBC counts for mice treated with G-CSF, AMD3100 or G-CSF+AMD3100.

FIG. 9 shows representative cell lines treated with the indicated reagents.

FIG. 10 shows the effect of different mobilization agents on cell lines.

FIG. 11 shows the effect of mobilization agents on murine peripheral blood WBC count.

FIG. 12 shows murine peripheral blood WBC count 5-7 days after treatment with mobilization agents.

FIG. 13 shows CFUs after treatment with mobilization agents.

FIG. 14 shows CFUs after treatment with mobilization agents.

### DEFINITIONS

To facilitate an understanding of the present invention, a number of terms and phrases are defined below:

As used herein, the term “cell” refers to any eukaryotic or prokaryotic cell (e.g., bacterial cells such as E. coli, yeast cells, mammalian cells, avian cells, amphibian cells, plant cells, fish cells, and insect cells), whether located in vitro or in vivo.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

As used herein, the term “stem cell” refers to self-renewing multipotent cells that are capable of giving rise to more stem cells, as well as to various types of terminally differentiated cells.

As used herein, the term “stem cell mobilization composition” refers to a composition comprising one or more reagents that mobilize stem cells from the bone marrow or other location into circulation (e.g., peripheral blood circulation). In some embodiments, stem cell mobilization compositions comprise one or more of cytokines (e.g., G-CSF), immunostimulants (e.g., Plerixafor) and neuropeptides (e.g., substance P).

As used herein, the terms “host” and “subject” refer to any warm blooded mammal, including, but not limited to, humans, non-human primates, rodents, and the like. Typically, the terms “host,” “subject” and “patient” are used interchangeably herein in reference to a human subject.

As used herein, the terms “defective tissues” and “defective cells” refer to tissues and cells that are marked by subnormal structure, function, or behavior. Defects responsible for the defective tissues and cells include known or detectable defects, as well as, unknown or undetectable defects.

As used herein, the term “neural defect” or “neurological disorder” refers to a defect involving or relating to the nervous system (including central and peripheral nervous systems). Some neural defects are caused by injury to the nervous system or defective tissues or cells of the nervous system, while other defects are caused by injury to cells that affect the nervous system or defective tissues or cells that affect the nervous system. As used herein, the term “neurally defective mammal” refers to a mammal having one or more neural defects. When a neural defect is “ameliorated,” the condition of the host is improved. For example, amelioration can occur when defective tissue is returned partially or entirely to a normal state. However, amelioration can also occur when tissue remains subnormal, but is otherwise altered to benefit the host.

As used herein, the term “degenerative disease” refers to a disease or disorder characterized by a decrease in function or degradation of normally functional tissues or organs.

As used herein, the term “non-human animals” refers to all non-human animals. Such non-human animals include, but are not limited to, vertebrates such as rodents, non-human primates, ovines, bovines, ruminants, lagomorphs, porcines, caprines, equines, canines, felines, ayes, etc.

The term “isolated” when used in relation to a cell (e.g., a stem cell or stem cell precursor), as in “an isolated cell” or “isolated cells” refers to cells that are separated and enriched in a sample so as to remove the isolated cell(s) from other cells with which it is ordinarily associated in its natural environment. For example, isolated stem cells are stem cells that are removed from their natural environment and enriched in a sample, such that the sample housing the stem cells contains a higher percentage of stem cells than a corresponding sample found in a tissue in its natural environment. The degree of enrichment can be defined relative to the source material (e.g., 10 fold enrichment). In some embodiments, a cell is isolated to a degree by which it is the prevalent cell type (i.e., most common) in the sample.

The term “sample” in the present specification and claims is used in its broadest sense. On the one hand it is meant to include a specimen or culture. On the other hand, it is meant to include both biological and environmental samples. A sample may include a specimen of synthetic origin.

Biological samples may be animal, including human, fluid (e.g., blood, serum, plasma, saliva, urine, etc.), solid (e.g., stool) or tissue, as well as liquid and solid food and feed products and ingredients such as dairy items, vegetables, meat and meat by-products, and waste. These examples are not to be construed as limiting the sample types applicable to the present invention.

The terms “test compound” and “candidate compound” refer to any chemical entity, pharmaceutical, drug, and the like that is a candidate for use to treat or prevent a disease, illness, sickness, or disorder of bodily function. Test compounds comprise both known and potential therapeutic compounds.

As used herein, the term “purified” or “to purify” refers to the removal of components (e.g., contaminants) from a sample.

As used, the term “eukaryote” refers to organisms distinguishable from “prokaryotes.” It is intended that the term encompass all organisms with cells that exhibit the usual characteristics of eukaryotes, such as the presence of a true nucleus bounded by a nuclear membrane, within which lie the chromosomes, the presence of membrane-bound organelles, and other characteristics commonly observed in eukaryotic organisms. Thus, the term includes, but is not limited to such organisms as fungi, protozoa, and animals (e.g., humans).

As used herein, the term “administration” refers to the act of giving a drug, prodrug, or other agent, or therapeutic treatment to a subject. Exemplary routes of administration to the human body can be through the eyes (ophthalmic), mouth (oral), skin (transdermal, topical), nose (nasal), lungs (inhalant), oral mucosa (buccal), ear, by injection (e.g., intravenously, subcutaneously, intratumorally, intraperitoneally, etc.), and the like.

As used herein, the term “co-administration” refers to the administration of at least two agents or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art.

As used herein, the term “pharmaceutical composition” refers to the combination of an active agent with a carrier, inert or active, making the composition especially suitable for therapeutic use.

The terms “pharmaceutically acceptable” or “pharmacologically acceptable”, as used herein, refer to compositions that do not substantially produce adverse reactions, e.g., toxic, allergic, or immunological reactions, when administered to a subject.

### DETAILED DESCRIPTION OF THE INVENTION

The present invention relates to mobilization of stem cells. In particular, the present invention relates to growth factor-induced mobilization of stem cells in vivo for collection or tissue repair.

Many diseases result from damage to tissues due to a range of factors from diabetes due to autoimmune effects to heart disease from blocked vessels. The use of cellular products to repair damaged tissues has gained momentum in recent years with approaches being explored for transplantation of islet cells in diabetic patients and injection of bone marrow (BM) derived cells, including mononuclear cells and mesenchymal stem cells (MSCs) in cardiac patients. Successful cellular therapy has been utilized for more than 50 years in bone marrow transplantation (BMT) with cellular products infused into patients to restore blood cell production (hematopoietic tissue) after myeloablative high dose chemotherapy. Transplantation of stem cells and other cellular products in BMT patients has provided evidence of the complexity of the tissue regeneration and demonstrated the requirement of appropriate long term-engraftment of stem cells and the role of the microenvironment or stroma to support homeostasis and differentiation of stem cells through production of growth factors. In addition, the BMT field has developed methods for mobilization of stem cells and progenitor cells into the circulation for ease of collection providing cellular grafts with enhanced engraftment potential compared to BM derived cells. Based upon the mobilization methods developed for BMT, researchers explored the potential of using mobilized peripheral blood cells (mPBCs) as a source of stem cells for repair of myocardial ischemia. The clinical trials performed to date with mPBCs have injected the cells into the myocardium and demonstrated modest improvements in cardiac function. Despite early results in mice using mobilization of stem cells into the peripheral circulation to repair cardiac ischemia, clinical applications using mobilization as a method to deliver stem cells or other therapeutic cells for tissue repair have not been successful.

A number of clinical trials are in progress using BM derived cells and in particular MSCs are being evaluated as a potential cell source for cardiac repair. MSCs are a multipotent cell of the BM stroma that are typically isolated based upon adherence to standard tissue culture flasks. Although these cellular therapy trials have exciting clinical data there are limitations to these approaches. Generation of autologous cells requires invasive techniques to harvest BM or other tissue samples; there is often a significant time delay in delivery due to the need for culture expansion of cells; the delivery of cellular products often requires an invasive technique (e.g., catheter injection); and the therapy may be limited to a single treatment.

The challenge for cellular therapy for regenerative medicine is to deliver the right cells to the damaged tissue and for these cells to integrate into the tissue to replace damaged cells. A major component of tissue repair is to reconstitute the micro environment that provides the niche for resident stem and progenitor cells and to produce growth factors and cytokines required for normal homeostasis. The mobilization of mesenchymal stem cells presents a simple methodology for reconstitution of the microenvironment. This enables resident stem cells in the tissues to repair ischemic tissue or facilitate delivery of tissue specific stem cells, such as cardiac stem cells, to provide the necessary environment to enable integration of these stem cells. Mobilization of MSCs represents an important component for tissue repair and highlights the limitations in current approaches.

Experiments conducted during the course of development of embodiments of the present invention demonstrated that G-CSF mobilized peripheral blood progenitor cell products (PBPC) from normal human donors fail to generate mesenchymal stem cells (MSCs) under culture conditions which routinely generate MSCs from BM. Further data demonstrated that the combination of growth factors, including, for example, AMD3100 plus G-CSF, can effectively mobilize MSCs in rodents and non human primates.

Accordingly, embodiments of the present invention provide compositions and methods for mobilizing stem cells (e.g., MSCs, hematopoietic stem cells, precursors thereof or combinations thereof) for collection (e.g., by apheresis) for reinfusion or to provide circulating stem cells for tissue repair thus providing a non-invasive delivery procedure that overcomes many of the obstacles of prior procedures.

### I. Mobilization Reagents

As described above, embodiments of the present invention provide mobilization reagents and mobilization compositions comprising the reagents for mobilizing stem cells or stem cell precursors. In some embodiments, mobilization reagents comprise a combination of growth factors such as, for example, a cytokine or colony stimulating factor and/or one or more additional mobilization reagents (e.g., neuropeptides, immunostimulants and the like).

In some embodiments, the cytokine is Granulocyte colony-stimulating factor (G-CSF or GCSF). In some embodiments, the G-CSF is recombinant human G-CSF (rhG-CSF). G-CSF is commercially available (e.g., Neupogen (Amgen, Thousand Oaks, Calif.) and Leukine (Genzyme, Cambridge, Mass.)). In other embodiments, additional cytokines or growth factors are utilized.

In some embodiments, an immunostimulant (e.g., Plerixafor (AMD3100); Genzyme) is administered. Plerixafor is a hematopoietic stem cell mobilizer with a chemical name 1,1′-[1,4-phenylenebis (methylene)]-bis-1,4,8,11-tetraazacyclotetradecane. It has the molecular formula C₂₈H₅₄N₈. The molecular weight of plerixafor is 502.79 g/mol. The structural formula is provided below

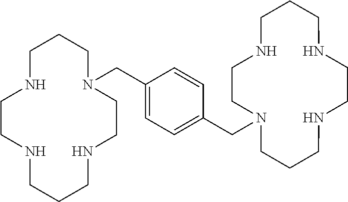

(See e.g., U.S. Pat. Nos. 5,583,131 and 6,987,102; each of which is herein incorporated by reference in its entirety). Additional active agents are contemplated to be within the scope of embodiments of the present invention.

In some embodiments, at least one additional reagent is administered in combination with the above. For example, in some embodiments, Substance P is utilized (See e.g., US 20060127373 and Hong et al., Nature Medicine, 15:425 (2009), each of which is herein incorporated by reference in its entirety). Substance P(SP) is an undecapeptide that functions as a neurotransmitter and as a neuromodulator. The deduced amino acid sequence of substance P is as follows: Arg Pro Lys Pro Gln Gln Phe Phe Gly Leu Met (SEQ ID NO:1).

As described, in some embodiments, combinations of two or more stem cell mobilization reagents are administered together or separately to a subject. In some embodiments, it is contemplated that the agents (e.g., G-CSF and Plerixafor) act synergistically to mobilize stem cells (e.g., MSCs or other non-hematopoietic stem cells).

Embodiments of the present invention further provide pharmaceutical compositions (e.g., comprising one or more of the therapeutic compounds described above). The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration.

Pharmaceutical compositions and formulations for topical administration (e.g., to tissues, wounds, organs, etc) may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Dosing is dependent on severity and responsiveness of the disease state or condition to be treated, with the course of treatment lasting from several days to several months, or until a cure is effected or a diminution of the disease state is achieved. In some embodiments, treatment is administered in one or more courses, where each course comprises one or more doses per day for several days (e.g., 1, 2, 3, 4, 5, 6) or weeks (e.g., 1, 2, or 3 weeks, etc.). In some embodiments, courses of treatment are administered sequentially (e.g., without a break between courses), while in other embodiments, a break of 1 or more days, weeks, or months is provided between courses. In some embodiments, treatment is provided on an ongoing or maintenance basis (e.g., multiple courses provided with or without breaks for an indefinite time period). Optimal dosing schedules can be calculated from measurements of drug accumulation in the body of the patient. The administering physician can readily determine optimum dosages, dosing methodologies and repetition rates.

In some embodiments, dosage is from 0.01 μg to 100 g per kg of body weight, and may be given once or more daily, weekly, monthly or yearly. For example, in some embodiments, Plerixafor is administered at a dosage of 0.1 to 1 mg/kg of body weight (e.g., 0.24 mg/kg). In some embodiments, G-CSF is administered at a dosage of 100 to 1000 μg or 1 to 50 μg/kg/day (e.g., 300 or 480 μg or 5 μg/kg/day). In some embodiments, substance P is administered at a dose of 0.05-1 nmole/g of body weight (e.g., 0.1 nmole/g of body weight). The treating physician can estimate repetition rates for dosing based on measured residence times and concentrations of the drug in bodily fluids or tissues.

### II. Uses

As described above, embodiments of the present invention provide compositions and methods for mobilizing stem cells and stem cell precursors (e.g., MSCs and/or non-hematopoietic stem cells). Such stem cells find use in a variety of clinical, pharmaceutical and research applications. For example, in some embodiments, the present invention finds use in the mobilization of stem cells and/or stem cell precursors for harvesting for generating allogeneic pheresis products. Such isolated cells find is in autologous and/or allogenic progenitor cell apheresis transplantation (stem cell transplant). Stem cell transplant finds use in the treatment of a variety of disease states and conditions, for example, replacement of dysfunctional bone marrow (e.g., in aplastic anemia) and in cancer treatment.

In some embodiments, stem cell mobilization finds use in the autologous treatment of diseases associated with tissue or neurological damage (e.g., tissue repair or anti-inflammatory action). A subject's own stem cells are mobilized in vivo, thus avoiding any potential complications of allogenic transplant and harvest. Examples of diseases that find use in such methods include, but are not limited to, cardiac disease (e.g., ischemic or degenerative cardiac disease), neurodegenerative diseases, inflammatory diseases and the like.

Neurodegenerative conditions (or disorders) include, but are not limited to, acute and chronic conditions, disorders or diseases of the central or peripheral nervous system. A neurodegenerative condition may be age-related, or it may result from injury or trauma, or it may be related to a specific disease or disorder. Acute neurodegenerative conditions include, but are not limited to, conditions associated with neuronal cell death or compromise including cerebrovascular insufficiency, e.g., due to stroke, focal or diffuse brain trauma, diffuse brain damage, spinal cord injury or peripheral nerve trauma, e.g., resulting from physical or chemical burns, deep cuts or limb severance. Examples of acute neurodegenerative disorders are: cerebral ischemia or infarction including embolic occlusion and thrombotic occlusion, reperfusion following acute ischemia, perinatal hypoxic-ischemic injury, cardiac arrest, as well as intracranial hemorrhage of any type (such as epidural, subdural, subarachnoid and intracerebral), and intracranial and intravertebral lesions (such as contusion, penetration, shear, compression and laceration), as well as whiplash and shaken infant syndrome. Chronic neurodegenerative conditions include, but are not limited to, Alzheimer's disease, Pick's disease, diffuse Lewy body disease, progressive supranuclear palsy (Steel-Richardson syndrome), multisystem degeneration (Shy-Drager syndrome), chronic epileptic conditions associated with neurodegeneration, motor neuron diseases including amyotrophic lateral sclerosis, degenerative ataxias, cortical basal degeneration, ALS-Parkinson's-Dementia complex of Guam, subacute sclerosing panencephalitis, Huntington's disease, Parkinson's disease, synucleinopathies (including multiple system atrophy), primary progressive aphasia, striatonigral degeneration, Machado-Joseph disease/spinocerebellar ataxia type 3 and olivopontocerebellar degenerations, Gilles De La Tourette's disease, bulbar and pseudobulbar palsy, spinal and spinobulbar muscular atrophy (Kennedy's disease), primary lateral sclerosis, familial spastic paraplegia, Werdnig-Hoffmann disease, Kugelberg-Welander disease, Tay-Sach's disease, Sandhoff disease, familial spastic disease, Wohlfart-Kugelberg-Welander disease, spastic paraparesis, progressive multifocal leukoencephalopathy, depression, mania, epilepsy, familial dysautonomia (Riley-Day syndrome), and prion diseases (including, but not limited to Creutzfeldt-Jakob, Gerstmann-Sträussler-Scheinker disease, Kuru and fatal familial insomnia), demyelination diseases and disorders including multiple sclerosis and hereditary diseases such as leukodystrophies.

Stroke refers to any condition arising from a disruption, decrease, or stoppage of blood or oxygen flow to any part of the brain. “Ischemic stroke” refers to a stroke resulting from any disruption, decrease or stoppage in the blood supply to any part of the brain caused from any constriction or obstruction of the vasculature. The obstruction of vasculature may be either temporal or permanent. “Hemorrhagic stroke” refers a stroke resulting from any rupture in any of the vasculature of the brain. Examples of acute neurodegenerative disorders that include stroke or involve etiology or symptoms such as those observed with stroke are listed above, and include: cerebral ischemia or infarction including embolic occlusion and thrombotic occlusion, reperfusion following acute ischemia, perinatal hypoxic-ischemic injury, cardiac arrest, as well as intracranial hemorrhage of any type (such as epidural, subdural, subarachnoid and intracerebral), and intracranial and intravertebral lesions (such as contusion, penetration, shear, compression and laceration), as well as whiplash and shaken infant syndrome.

Other neurodegenerative conditions include tumors and other neoplastic conditions affecting the CNS and PNS. Though the underlying disease is considered proliferative (rather than neurodegenerative), surrounding tissues may be compromised. Other neurodegenerative conditions include various neuropathies, such as multifocal neuropathies, sensory neuropathies, motor neuropathies, sensory-motor neuropathies, infection-related neuropathies, autonomic neuropathies, sensory-autonomic neuropathies, demyelinating neuropathies (including, but not limited to, Guillain-Barre syndrome and chronic inflammatory demyelinating polyradiculoneuropathy), other inflammatory and immune neuropathies, neuropathies induced by drugs, neuropathies induced by pharmacological treatments, neuropathies induced by toxins, traumatic neuropathies (including, but not limited to, compression, crush, laceration and segmentation neuropathies), metabolic neuropathies, endocrine and paraneoplastic neuropathies, among others.

Other neurodegenerative conditions include dementias, regardless of underlying etiology, including age-related dementia and other dementias and conditions with memory loss including dementia associated with Alzheimer's disease, vascular dementia, diffuse white matter disease (Binswanger's disease), dementia of endocrine or metabolic origin, dementia of head trauma and diffuse brain damage, dementia pugilistica and frontal lobe dementia.

Examples of inflammatory diseases include, without limitation, myocardial infarction (MI), diabetes, stroke, Alzheimer's disease, multiple sclerosis, parkinsonism, nephritis, cancer, inflammatory diseases involving acute or chronic inflammation of bone and/or cartilage in a joint, anaphylactic reaction, asthma, conjunctivitis, systemic lupus erythematosus, pulmonary sarcoidosis, ocular inflammation, allergy, emphysema, ischemia-reperfusion injury, fibromyalgia, and inflammatory cutaneous disease selected from psoriasis and dermatitis, or an arthritis selected from rheumatoid arthritis, gouty arthritis, juvenile rheumatoid arthritis, and osteoarthritis.

In some embodiments, stem cell mobilization finds use in tissue repair (e.g., for regenerative medicine applications). It is contemplated that the compositions and methods find use in instances where tissue repair is needed or desired or reduction or prevention of tissue damage is needed or desired. Examples include, but are not limited to, protection or repair of tissues such as liver, kidney, neural, pulmonary, and skin, resulting from any cause (e.g., disease, tissue or organ transplant, etc.).

In some embodiments, stem cell mobilization finds use in decreasing tissue damage due to inflammatory responses (e.g., in myocardial infarction, graft versus host disease, wound healing, and stroke). Examples of diseases and conditions in which tissue damage occurs due to inflammatory responses include, but are not limited to, cardiovascular diseases (e.g., atherosclerosis, heart failure, cardiomyopathy, stroke, and cerebrovascular disease), diabetic complications (e.g., cardiomyopathy, atherosclerosis, chronic renal failure, retinopathy, sepsis neuropathy), chronic inflammatory disorders (e.g., inflammatory bowel disease, chronic obstructive pulmonary disease, rheumatoid arthritis, psoriasis, chronic pancreatitis, chronic inflammatory demyelinating polyneuropathy, chronic inflammatory connective tissue diseases), bone, muscular, and skeletal disease (e.g., osteoporosis, osteoarthritis, degenerative disc disease, muscular dystrophy), metabolic disorder complications (e.g., fatty liver disease, heart disease, type 2 diabetes, chronic kidney disease, sleep apnea), and neurological disorders (e.g., Alzheimer's, Parkinson's, amyotrophic lateral sclerosis, dementia).

In some embodiments, stem cell mobilization finds use in tissue regeneration and/or decreasing tissue damage following solid organ transplant (e.g., liver, kidney, lung, heart, pancreas, etc.), tissue transplants, or other allotransplantations.

In some embodiments, therapeutic or research treatments of a subject are coupled with one or more screening or diagnostic tests. In some embodiments, such tests are used to select a patient prior to treatment (e.g., as having a particular disease or conditions or signs or symptoms thereof). In some embodiments, such tests are used to monitor the results of a treatment (for example, to determine if treatment can be stopped, should be continued, should be changed, etc.). In some embodiments testing occurs one or more times before and/or after treatment. Testing may be conducted using any suitable approach and testing and or management or reporting of test results may employ a computer system, software, a database, or other components. In some embodiments testing comprises determining the location and/or status of a stem cell.

### EXPERIMENTAL

The following examples are provided in order to demonstrate and further illustrate certain preferred embodiments and aspects of the present invention and are not to be construed as limiting the scope thereof.

### Example 1

### Are MSCs or MSC Precursors Present in Peripheral Blood

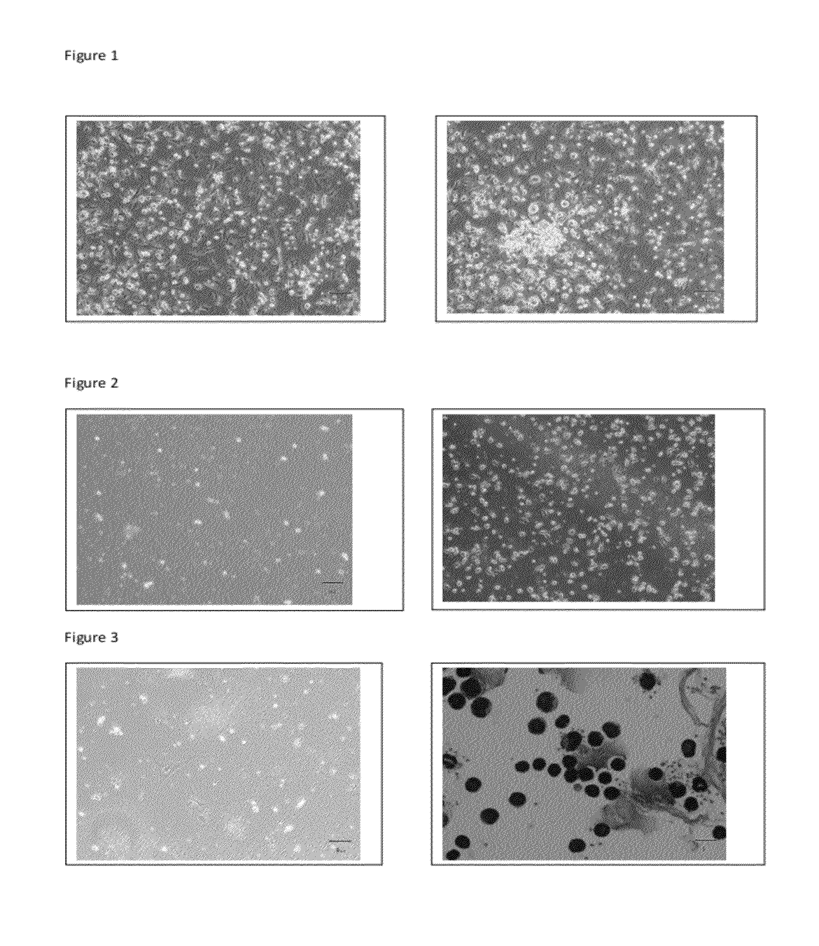

Recent studies demonstrating the improvement in cardiac function in patients experiencing an acute MI with injection of MSCs into the peripheral blood led to an investigation of whether normal peripheral blood (PB) or mobilized PB contains MSCs or CD271+ cells. Normal unmobilized PB and PB from normal donors mobilized with rhG-CSF was obtained and the potential of the MNCs to generate MSCs was evaluated. When placed in standard culture there was development of adherent cells that appeared like adipocyte cells and endothelial cells (FIG. 1). When passaged the adherent cells failed to continue to grow and there was no evidence of MSC formation in any PB samples tested. Some G-CSF mobilized PB products were selected for CD271+ cells but these cells failed to generate MSCs and upon flow analysis contained almost exclusively double positive cells for CD271+ and CD45+ cells. These results indicate that there are few if any MSCs or MSC precursors in steady state PB or G-CSF mobilized PB.

### Mobilization of Mice

To evaluate the potential of different mobilization agents, a mouse model of mobilization was used. Groups of mice (n=5) were treated with cytokines or mobilization agents and peripheral blood harvested into heparin containing tubes. The blood was treated with ammonium chloride to lyse the red cells and the white cells plated in 6 well plates treated with gelatin and evaluated for MSC formation at various time points.

Mobilization with rhG-CSF

Mice were injected intraperitoneally with 250 ug/kg of rhG-CSF (Amgen Inc, Thousand Oaks, Calif.) or normal saline for 5 days and PB harvested on the fifth day. The white cells were plated in media from Stem Cell Technologies (Vancouver, Canada) for culture of mesenchymal stem cells with murine supplements. Cultures of WBC from mice treated with saline failed to form adherent cells and contained few cells (FIG. 2). In contrast, cultures of WBC from rhG-CSF treated mice contained significantly more cells and formed adherent cells as shown in FIG. 2. The adherent cells failed to proliferate and resembled endothelial cells not MSCs.

Mobilization with Substance P

Mice (groups of 5 mice per treatment arm) were injected intraperitoneally with 10 μg/kg of Substance P (Sigma-Aldrich, St Louis, Mo.) or with 250 ug/kg of rhG-CSF for 4 days and PB harvested on the fifth day. The white cells were plated in media from Stem Cell Technologies (Vancouver, Canada) for culture of mesenchymal stem cells with murine supplements. Cultures of WBC from mice treated with SP generated adherent stromal cells after 5 to 7 days with several foci observed in 35 mm wells (FIG. 3). Cultures of WBC from rhG-CSF treated mice contained significantly more cells and formed adherent cells as described above with no stromal cell formation evident.

Cytopsin slides of the WBC from SP treated mice demonstrated an increase in blast like cells as demonstrated in FIG. 3.

Mobilization with the Combination of rhG-CSF and SP

It is contemplated that based upon the initial data shown above for mobilization of MSCs by SP, combining SP with rhG-CSF provides a synergistic increase of MSC precursor cells in the peripheral blood of treated animals. This was based upon the large number of cells migrating from the BM of mice treated with rhG-CSF to the peripheral circulation. It was contemplated that this increased trafficking of cell would similarly shift MSC precursor cells into the peripheral circulation at higher levels than SP treatment alone.

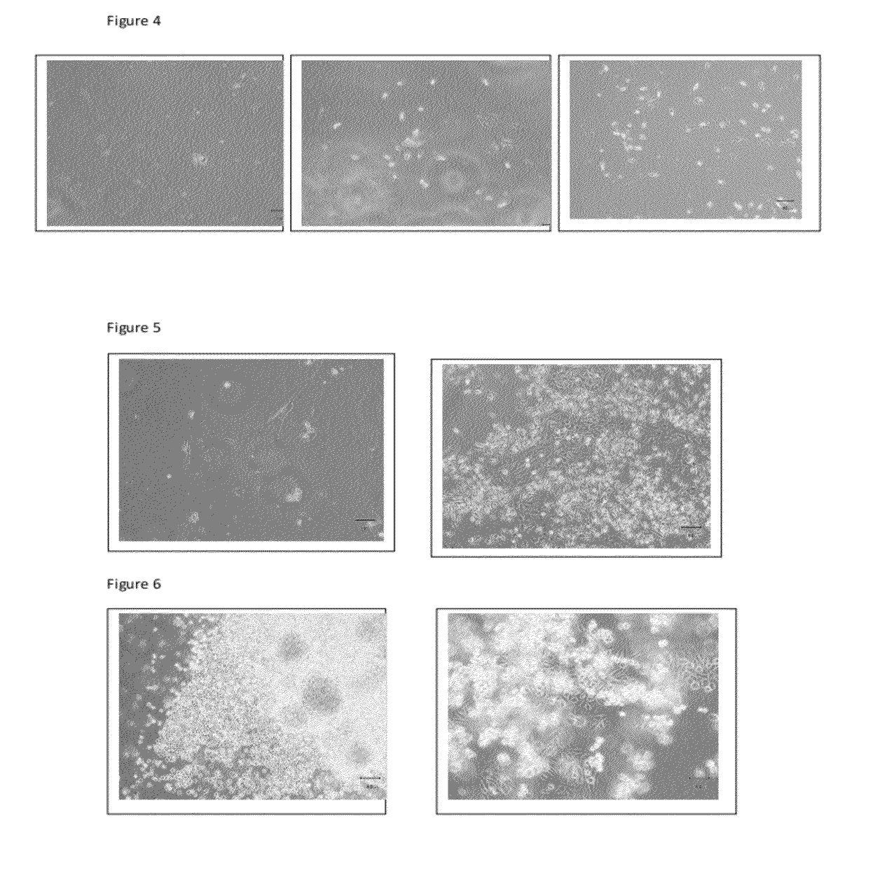

Mice (groups of 5 mice per treatment arm) were injected intraperitoneally with 10 μg/kg of Substance P (Sigma-Aldrich, St Louis, Mo.), with 250 μg/kg of rhG-CSF, or the combination of 10 μg/kg SP plus 250 μg/kg rhG-CSF for 4 days and PB harvested on the fifth day. The white cells were plated in media from Stem Cell Technologies (Vancouver, Canada) for culture of mesenchymal stem cells with murine supplements. Cultures of WBC from mice treated with SP generated a few adherent stromal cells after 5 to 7 days, while rhG-SCF resulted in one area with MSC cells while the combination of SP plus rhG-CSF resulted in many MSC foci observed in 35 mm wells. After 10 days of culture several MSC foci were observed in culture from SP treated mice, one foci from rhG-CSF treated mice and approximately 50% of the well contained MSC in SP plus rhG-CSF treated mice (FIG. 4). Between 2 and 3 weeks of culture the MSCs in cultures from SP+G-CSF approached confluency as shown in FIG. 5.

WBC from treated mice were also plated in standard hematopoietic progenitor colony forming assays. Colony formation from cultures of 200,000 cells per 35 mm petri dishes were scored at 10 days of culture. As shown in Table 1, all cultured contained colonies with similar number of GM-CFC for cells from rhG-CSF treated animals and rhG-CSF+SP treated animals.

TABLE 1
Colony Formation in Methycellulose Culture
After 10 Days of Incubation.

| WBC from treated mice | Number of GM-CFC (n = 3) |
|---|---|
| SP | 5 |
| rhG-CSF | 26 |
| rhG-CSF + SP | 22 |

The median colony number pre 200,000 cells plated is presented from 3 triplicate cultures containing 1 ml of methycellulose plus rmIL-3, rmGM-CSF, rrSCF and rhG-CSF.

Typical hematopoietic colonies formed in the cultures of WBC from rhG-CSF treated mice while colonies containing both hematopoietic and MSC cells formed in cultures of WBC from SP alone of rhG-CSF+SP treated mice (FIG. 6).

### Effect of SP+G-CSF on Peripheral Blood WBC Counts:

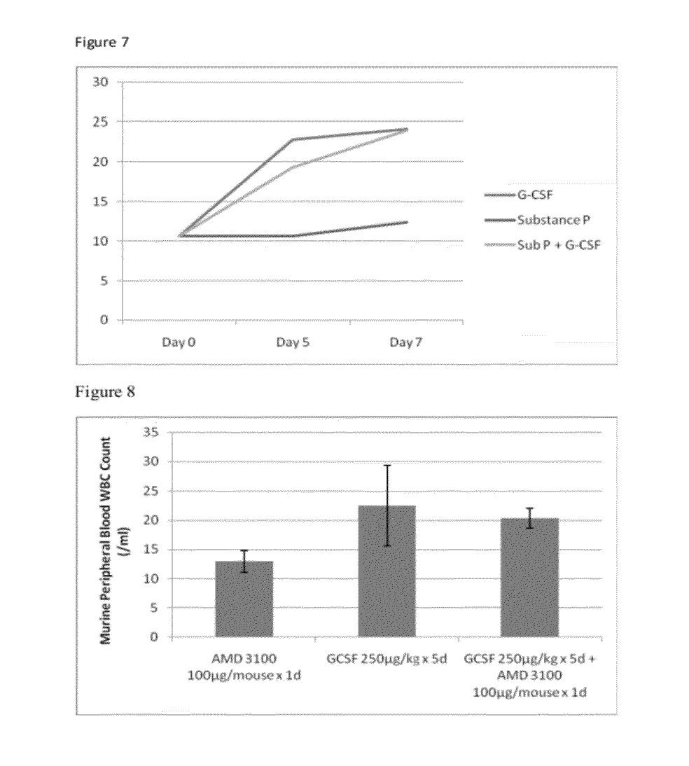

G-CSF mobilization increases the circulating WBC count in mice and humans with typical counts approximately 25,000 to 30,000 WBC/μl. The effects of the combination of SP+G-CSF on peripheral WBC in treated mice was evaluated. Treatment of mice with SP had no significant effect on WBC levels through the 7 day treatment (FIG. 7). The combination of SP+G-CSF resulted in equivalent WBC levels as G-CSF alone.

Mobilization with the Combination of rhG-CSF and AMD3100

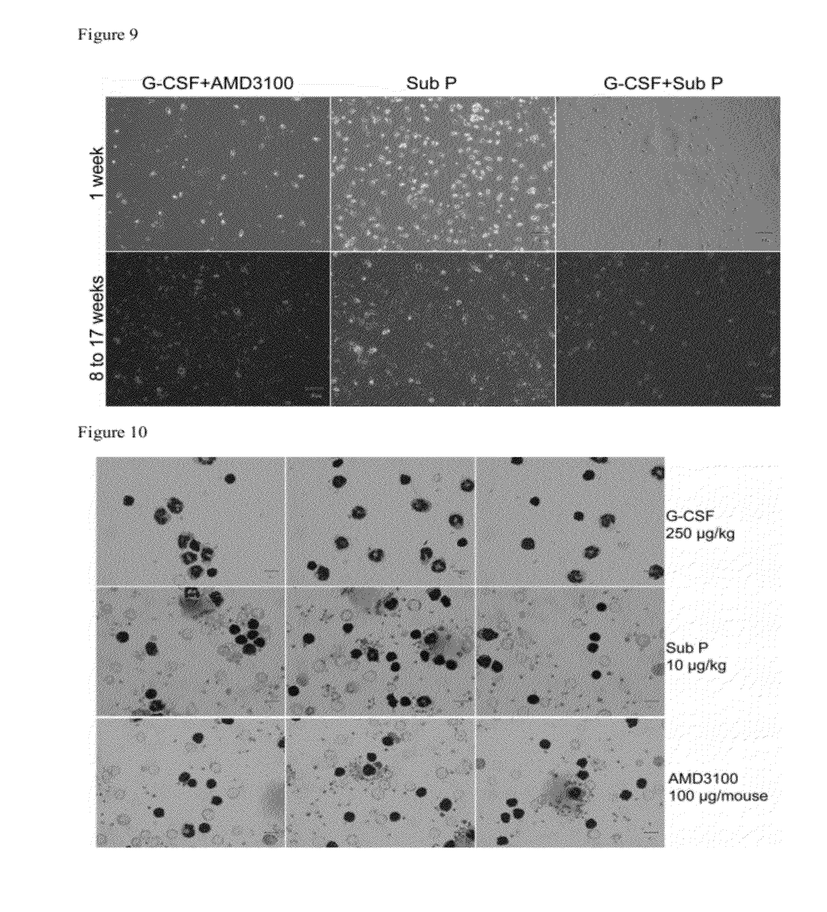

12 cell lines were established. Representative cell lines are shown in FIG. 9. The effects of different mobilization agents used is shown in FIG. 10.

The different effects on peripheral WBC count of mobilization agents used were established.

G-CSF±AMD 3100

Group 1: AMD 3100 100 μg/mouse×1d
Group 2: GCSF 250 m/kg×5d
Group 3: GCSF 250 μg/kg×5d+AMD 3100 100 μg/mouse×1d

### One-way ANOVA: sig. <0.01

Post Hoc tests: LSD:
AMD 3100 100 μg/mouse×1d vs. GCSF 250 μg/kg×5d: sig. <0.01
AMD 3100 100 μg/mouse×1d vs. GCSF 250 μg/kg×5d+AMD 3100 100 μg/mouse×1d: sig. <0.05

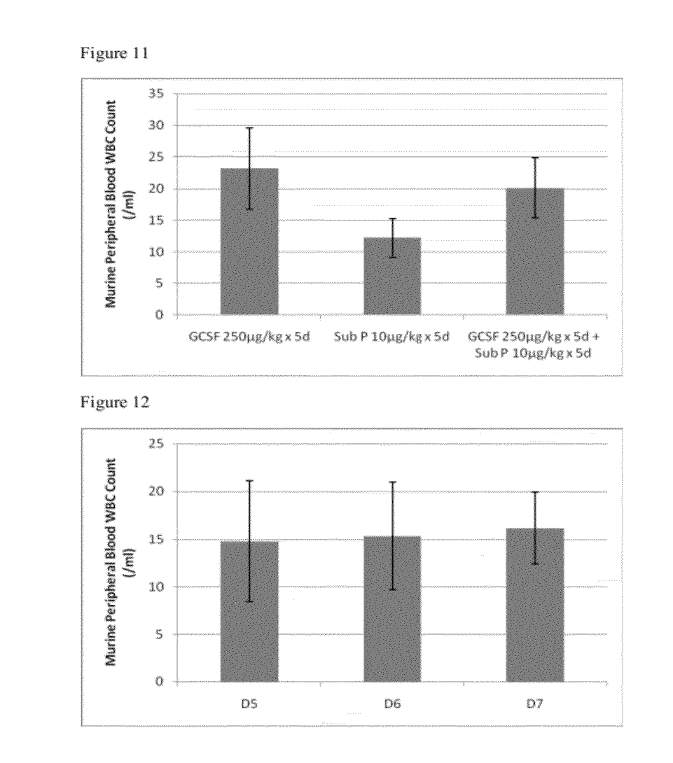

G-CSF±Sub P Group 1: GCSF 250 μg/kg×5d (results shown in FIG. 11)

Group 2: Sub P 10 μg/kg×5d
Group 3: GCSF 250 m/kg×5d+Sub P 10 μg/kg×5d

### One-way ANOVA: sig. <0.05

Post Hoc tests: LSD:
Sub P 10 μg/kg×5d vs. GCSF 250 μg/kg×5d: sig. <0.01
Sub P 10 μg/kg×5d vs. GCSF 250 μg/kg×5d+Sub P 10 μg/kg×5d: sig. <0.05

It was demonstrated that during the course of 5-7 days mobilization, the WBC count is not time-dependent (FIG. 12).

Agents used: GCSF 250 m/kg×5d+Sub P 100 μg/kg×5d

### One-way ANOVA: sig. 0.917

A CFU assay was performed: 200,000 MNC from murine PB were plated in methylcellulose (M4230, StemCell Technologies) supplemented with 100 ng/ml rr SCF, 100 ng/ml rm IL-3, 100 ng/ml rh IL-6, 100 ng/mL rh G-CSF and 100 ng/mL rm GM-CSF. Hematopoietic colonies (committed colony-forming cells granulocyte-macrophage, CFU-GM) were scored after 14 days of culture in 5% CO2 at 37° C. according to the established criteria. Colonies that reached greater than 0.5 mm in size after 14 days were scored as high-proliferative potential colonyforming cells (HPP-CFC).

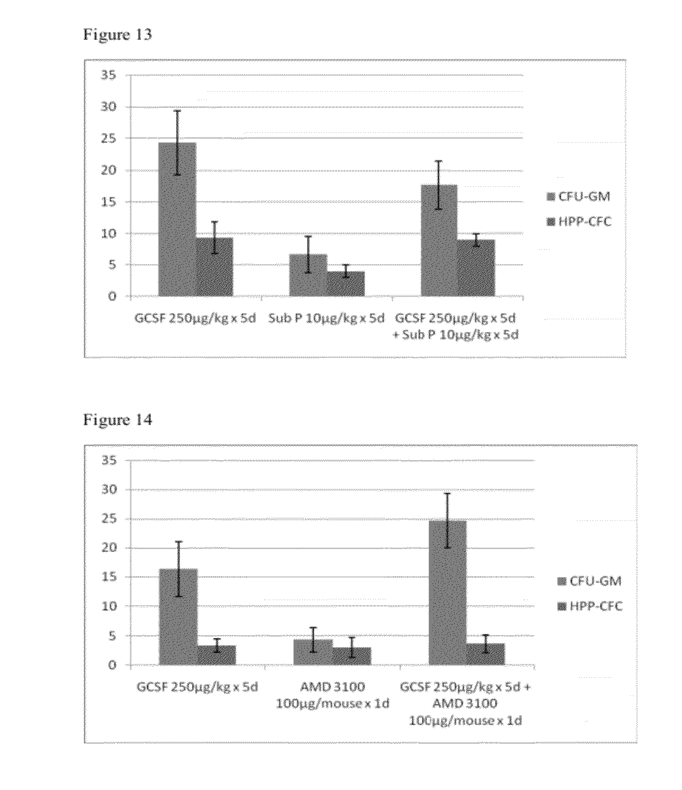

(1) G-CSF±Sub P (FIG. 13)

Group 1: GCSF 250 m/kg×5d
Group 2: Sub P 10 μg/kg×5d
Group 3: GCSF 250 μg/kg×5d+Sub P 10 μg/kg×5d

### One-way ANOVA: CFU-GM: sig. <0.01

Post Hoc tests: LSD:
Sub P 10 μg/kg×5d vs. GCSF 250 μg/kg×5d: sig. <0.01
Sub P 10 μg/kg×5d vs. GCSF 250 μg/kg×5d+Sub P 10 μg/kg×5d: sig. <0.05

### One-way ANOVA: HPP-CFC: sig: <0.05

Sub P 10 μg/kg×5d vs. GCSF 250 μg/kg×5d: sig. <0.01
Sub P 10 μg/kg×5d vs. GCSF 250 μg/kg×5d+Sub P 10 μg/kg×5d: sig. <0.05

(2) G-CSF±AMD 3100 (FIG. 14)

Group 1: GCSF 250 μg/kg×5d
Group 2: AMD 3100 100 μg/mouse×1d
Group 3: GCSF 250 μg/kg×5d+AMD 3100 100μg/mouse×1d

### One-way ANOVA: CFU-GM: sig. <0.01

Post Hoc tests: LSD:
AMD 3100 100 μg/mouse×1d vs. GCSF 250 μg/kg×5d: sig. <0.05
AMD 3100 100 m/mouse×1d vs. GCSF 250 μg/kg×5d+AMD 3100 100 μg/mouse×1d: sig. <0.01
GCSF 250 μg/kg×5d vs. GCSF 250 μg/kg×5d+AMD 3100 100 m/mouse×1d: sig. <0.05

### One-way ANOVA: HPP-CFC: sig: 0.864

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be within the scope of the following claims.

We claim:

1. A method of mobilizing stem cells, comprising:
administering a stem cell mobilization composition, comprising: a) at least one cytokine; and b) Plerixafor to a subject under conditions such that said subject mobilizes circulating stem cells.

2. The method of claim 1, wherein said cytokine is granulocyte colony-stimulating factor (G-CSF).

3. The method of claim 2, wherein said G-CSF is recombination human G-CSF (rhG-CSF).

4. The method of claim 1, wherein said composition further comprises at least one additional stem cell mobilization reagent.

5. The method of claim 4, wherein said additional stem cell mobilization reagent is a neuropeptide.

6. The method of claim 5, wherein said neuropeptide is substance P.

7. The method of claim 6, wherein said substance P has the amino acid sequence of SEQ ID NO:1.

8. The method of claim 1, wherein said composition comprises at least one pharmaceutically acceptable carrier.

9. The method of claim 1, further comprising the step of isolating said stem cells from said subject.

10. The method of claim 1, wherein said stem cells are mesenchymal stem cells.

11. The method of claim 1, wherein said subject exhibits symptoms of a neurodegenerative, cardiac, liver, kidney, lung or inflammatory disease.

12. The method of claim 11, wherein said administration results in a decrease in said symptoms.

13. The method of claim 1, wherein said subject has undergone or is undergoing an organ transplant.

14. The method of claim 13, wherein said organ is selected from the group consisting of kidney, liver, lung, pancreas and heart.
